(19) United States
(12) Patent Application Publication
(10) Pub. No.: US 2004/0105827 A1
(43) Pub. Date: Jun. 3, 2004

(54) Use for make-up in particular of a cosmetic composition having a continuous hydrophilic comprising a multilayer goniochromatic pigment

(76) Inventors: Sabine Grimm, Chatenay Malabry (FR); Jean-Christophe Simon, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/148,580

(22) PCT Filed: Oct. 3, 2001
(86) PCT No.: PCT/FR01/03050

(30) Foreign Application Priority Data
Oct. 3, 2000 (FR) 00/12602

(51) Int. Cl. A61K 7/021

(52) U.S. Cl. 424/63

(57) ABSTRACT

The invention relates to the use of a cosmetic composition with a hydrophilic continuous phase comprising a goniochromatic pigment with an interferential multilayer structure as make-up.

The present invention relates to the use of a cosmetic composition, in particular a make-up composition, comprising a goniochromatic pigment with an interferential multilayer structure.

Make-up compositions, such as free or compact powders, foundations, blushers, eyeshadows or lipsticks, are composed of an appropriate vehicle and of various colouring agents intended to confer a certain colour on the compositions, before and/or after their application to the skin (including the lips) and/or the superficial body growths.

A fairly restricted range of colouring agents, in particular pigments, such as lakes, inorganic pigments or pearlescent pigments, is currently used to create colours. Lakes make it possible to obtain vivid colours but are generally unstable to light, to temperature and to pH. Some also exhibit the disadvantage of staining the skin in an unsightly way after application, by discharge of the colorant. Inorganic pigments, in particular inorganic oxides, are, in contrast, very stable but give rather dull and pale colours. Pearlescent pigments make it possible to obtain varied but never intense colours with iridescent but generally fairly weak effects, and in particular the colour effect is mainly visible along only one given angle corresponding to the specular reflection. For example, patent WO-A-96/03962 discloses a cosmetic composition in the form of an emulsion comprising a silicone oil, an “interferential” pigment of platelet type composed of a support such as mica coated with a given layer of titanium oxide with a given thickness, and a pigment based on iron oxide. Such a composition gives a shade along a given angle and is not goniochromatic.

On the other hand, it is advantageous to find compositions which give a volume effect once they have been applied to the skin, either for the lips (this is then described as “pulping”) or for the skin of the face (this is then described as “morphing”). Indeed, consumers are interested in cosmetic compositions which make it possible to remodel the face and/or the lips, so as to change the physical appearance and/or to give an aesthetic effect different from that of the face and/or the lips without make-up. No effective solution yet exists for satisfying this expectation.

One of the objects of the present invention is to provide the use of a cosmetic composition which exhibits effects, in particular aesthetic, goniochromatic and volume effects. Such effects are visible in particular for foundations, lipsticks, mascaras or eyeliners. As regards the skin, such “morphing” effects, allow sculpturing of the skin to which it is applied, by the possibility of alternation of darker regions and of lighter regions.

The invention therefore relates to the use, in particular as a make-up, of an interferential goniochromatic pigment in a cosmetic composition in order to give a volume effect, the said cosmetic composition containing a hydrophilic continuous phase and comprising at least one goniochromatic pigment with an interferential multilayer structure and a cosmetically acceptable medium containing a hydrophilic continuous phase, the said medium comprising at least one hydrophilic substance, which is at least partially miscible with or soluble in water, which may be liquid, pasty or solid at ambient temperature and at atmospheric pressure.

In a preferred embodiment, the said composition comprises from 0.01 to 5% of pigment other than the goniochromatic pigment with interferential multilayer structure, by weight with respect to the total weight of the composition.

Such a composition advantageously exhibits a goniochromatic effect, that is to say that it is capable of producing different colours depending upon the incidence of the light and the angle of observation, this being achieved mainly by the presence of a goniochromatic pigment with an interferential multilayer structure, the specific goniochromatic effect of which is not masked within the composition. The invention thus makes it possible to obtain a novel cosmetic effect, in particular a make-up effect: the colours appear and disappear according to the movements of the person wearing the make-up and the light which illuminates her. The term “colour” is understood to mean any colour in the visible spectrum. The make-up thus appears “living”, advantageously creating a volume effect.

The cosmetic composition can also advantageously exhibit a soft-focus effect, that is to say an effect which conceals the microreliefs of the skin (including the lips).

The term “pigment” is understood to mean particles which are insoluble in the medium constituting the cosmetic composition, that is to say in the dispersed or solid state in one of the phases of the said medium, and which are used to colour (creation or modification of shades of colour) and/or to opacify the said composition.

The term “goniochromatic pigment with interferential multilayer structure”, known as interferential goniochromatic pigment according to the invention, is understood to mean a pigment with an at least bilayer structure, the said layers being such that they make possible the creation of an effect of colour by interference of the light rays, which diffract and scatter differently according to the layers. Thus, such pigments can exhibit colours which vary according to the angle of observation and the incidence of the light and can confer iridescent highlights.

The multilayer structure can comprise at least two layers, each layer, independently or not independently of the other layer(s), being made of at least one material chosen from the group consisting of the following materials:

MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The goniochromatic pigment with an interferential multilayer structure according to the invention is preferably chosen from the group consisting of the following commercial goniochromatic pigments: Infinite Colors from Shiseido, Sicopearl Fantastico from BASF, Colorstream from Merck, Xirallic from Merck, Colorglitter from 3M and Chromaflair from Flex.

Consequently, the multilayer structure can be essentially inorganic or organic. Different colours, and therefore different effects, are obtained according to the thickness of each of the various layers.

The pigments with an interferential multilayer structure according to the invention are in particular those disclosed in the following documents: U.S. Pat. No. 3,438,796, EP-A-227 423, U.S. Pat. No. 5,135,812, EP-A-170 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A-4 225 031, WO 95/17479 (BASF) or DE-A-196 14 637, and their combinations. They are provided in the form of glitter, with a metallic colour.

For example, the interferential multilayer structure is chosen from the group consisting of the structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MOS₂/SiO₂/mica-oxide/SiO₂/MOS₂; Fe₂O₃/SiO₂/mica-oxide/SiO₂/Fe₂O₃.

Generally, the structure is composed of an alternation of layers with a low optical index and a high optical index.

The interferential pigment used according to the invention may be incorporated in a cosmetic, in particular a make-up, composition with a hydrophilic continuous phase in an amount which can be easily determined by a person skilled in the art on the basis of his general knowledge and which can in particular range from 0.01 to 50%, preferably from 0.5 to 25%, by weight with respect to the total weight of the composition. Even at a high concentration, this pigment has virtually no destructuring effect on the composition.

The composition used according to the invention can be provided in the form of a product to be applied to the skin (including the lips) and/or the superficial body growths of human beings, in particular the skin, both of the human body and of the face, the hair, the eyelashes and the eyebrows. It thus comprises a cosmetically acceptable medium, that is to say a medium compatible with all keratinous substances, such as the skin, both of the human body and of the face, the hair, the eyelashes and the eyebrows.

According to the invention, this medium contains a hydrophilic continuous phase, that is to say at least one hydophilic substance, which is at least partially miscible with or soluble in water, which may be liquid, pasty or solid at ambient temperature (generally 25° C.) and at atmospheric pressure (760 mmHg, that is 1.013×10⁵ Pa) . In particular, this medium can comprise or be provided in particular in the form of a suspension, dispersion or solution in water or an aqueous/alcoholic medium which is optionally thickened, indeed even gelled; oil-in-water (O/W) or multiple (W/O/W) emulsion, in the form of a cream, paste or even solid; aqueous or aqueous/alcoholic gel or hydrophilic foam; emulsified gel; dispersion of vesicles, in particular of ionic or nonionic lipids; two-phase or multiphase lotion; or spray. A person skilled in the art can choose the appropriate pharmaceutical dosage form, and its method of preparation, on the basis of his general knowledge, taking into account, on the one hand, the nature of the constituents used, in particular their solubility in the vehicle, and, on the other hand, the application envisaged for the composition.

The composition used according to the invention therefore comprises the said cosmetically acceptable medium which comprises a hydrophilic continuous phase which can contain water or a mixture of water and at least one hydrophilic organic solvent such as, for example, an alcohol chosen in particular from the group consisting of linear or branched lower monoalcohols having from 2 to 5 carbon atoms, such as ethanol or propanol, polyols such as glycerol, diglycerol, propylene glycol, sorbitol, panthenol, pentylene glycol, polyethylene glycols, and their mixtures. This continuous phase can represent from 0.5 to 99.99% by weight with respect to the total weight of the composition. It can additionally comprise at least one hydrophilic C₂ ether and at least one hydrophilic C₂-C₄ aldehyde.

When the medium used according to the invention is provided in the form of an emulsion, the composition according to the invention can optionally comprise, in addition, a surfactant, preferably in an amount of 0 to 30%, preferably of 0.01 to 30%, by weight with respect to the total weight of the composition.

According to the application envisaged, the composition can comprise, in addition, at least one film-forming polymer, for example chosen from the group consisting of polyurethanes, polyacrylics, polyurethane and polyacrylic hybrids, polyesters, nitrocellulose, hydrocarbon-based and/or silicone resins, and their blends. This is in particular the case when it is desired to prepare a composition of mascara or eyeliner type or a hair composition of lacquer type. The polymer can be dissolved or dispersed in the cosmetically acceptable medium and can optionally be used in combination with at least one coalescence agent and/or at least one plasticizer.

The composition used according to the invention can also comprise a fatty phase, which contains in particular at least one fatty substance which is liquid at ambient temperature and at atmospheric pressure and/or at least one fatty substance which is solid at ambient temperature and at atmospheric pressure.

The fatty substance which is liquid at ambient temperature and at atmospheric pressure, often known as oil, which can be used in the invention is chosen, for example, from the group consisting of: hydrocarbon-based oils of animal origin, such as perhydrosqualene; vegetable hydrocarbon-based oils, such as liquid triglycerides of fatty acids comprising 4 to 10 carbon atoms, such as triglycerides of heptanoic acid or octanoic acid, or sunflower, maize, soybean, grape seed, sesame, apricot, macadamia, castor or avocado oils, triglycerides of caprylic/capric acids, jojoba oil or karite butter oil; linear or branched hydrocarbons of mineral or synthetic origin, such as liquid paraffins and their derivatives, petroleum jelly, polydecenes or hydrogenated polyisobutene, such as Parleam; isododecane; synthetic esters and ethers, in particular of fatty acids, such as, for example, Purcellin oil, isopropyl myristate, 2-ethylhexyl palmitate, 2-octyldodecyl stearate, 2-octyldodecyl erucate, isostearyl isostearate or isononyl isononanoate; hydroxylated esters, such as isostearyl lactate, octyl hydroxystearate, octyldodecyl hydroxystearate, diisostearyl malate or triisocetyl citrate; heptanoates, octanoates or decanoates of fatty alcohols; polyol esters, such as propylene glycol dioctanoate, neopentyl glycol diheptanoate, diethylene glycol diisononanoate and pentaerythritol esters; fatty alcohols having from 12 to 26 carbon atoms, such as octyldodecanol, 2-butyloctanol, 2-hexyldecanol, 2-undecylpentadecanol or oleyl alcohol; fluorinated oils which are partially hydrocarbon-comprising and/or silicone-comprising; silicone oils, such as volatile or nonvolatile and linear or cyclic polymethylsiloxanes (PDMSs) which are liquid or pasty at ambient temperature and at atmospheric pressure, such as cyclomethicones, dimethicones, optionally comprising a phenyl group, such as phenyl trimethicones, phenyltrimethylsiloxydiphenylsiloxanes, diphenylmethyldimethyltrisiloxanes, diphenyl dimethicones, phenyl dimethicones or polymethylphenylsiloxanes; and their mixtures.

These oils can represent from 0 to 90%, preferably from 0.01 to 85%, by weight with respect to the total weight of the fatty phase.

The optional fatty phase of the composition used according to the invention can advantageously comprise a fatty phase which is solid or pasty at ambient temperature and at atmospheric pressure comprising at least one compound chosen from the group consisting of waxes, pasty fatty substances, gums and their mixtures. The waxes can be hydrocarbon-based, fluorinated and/or silicone waxes and can be of vegetable, mineral, animal and/or synthetic origin. In particular, the waxes exhibit a melting temperature of greater than approximately 25° C., preferably of greater than approximately 45° C., and the pasty fatty substances exhibit a melting temperature from approximately 25° C. to approximately 45° C.

The wax which can be used in the composition of the invention is, for example, chosen from the group consisting of beeswax, carnauba wax, candelilla wax, jojoba wax (hydrogenated or nonhydrogenated), paraffin wax, microcrystalline waxes, ceresin or ozokerite; synthetic waxes, such as polyethylene waxes or Fischer-Tropsch waxes; silicone waxes, such as alkyl or alkoxy dimethicones having from 16 to 45 carbon atoms; and their mixtures.

The gum which can be used according to the invention is generally chosen from the group consisting of PDMSs with a high molecular weight, cellulose gums, polysaccharides and their mixtures, and the pasty substance which can be used according to the invention is generally chosen from the group consisting of hydrocarbon-based compounds, such as lanolins and their derivatives, PDMSs and their mixtures.

The nature and the amount of the solid substances depend on the mechanical properties and textures desired. By way of indication, the composition can comprise from 0 to 50%, preferably from 0.01 to 40%, more preferably still from 0.1 to 30%, by weight with respect to the total weight of the composition, of at least one compound chosen from the group consisting of waxes, pasty fatty substances, gums and their mixtures.

The method for preparing the cosmetic composition used according to the invention, comprising a fatty phase, a hydrophilic phase and a goniochromatic pigment with an interferential multilayer structure, comprises:

1. the preparation of the generally aqueous hydrophilic phase and heating the said phase up to the temperature at which the said phase boils,

2. the preparation of the fatty phase, and the dispersion of the interferential goniochromatic pigment in the said phase, which is made liquid, if necessary, by heating,

3. the preparation of the emulsion by bringing the fatty phase thus produced in step 2 into contact with the hydrophilic phase thus produced in step 1, for example by means of an appropriate turbine such as the Mortiz microdispersing device, and

4. maintaining the stirring until there has been a return to ambient temperature, for example by the use of a turbine and then of paddles.

In particular, the method of manufacture makes it possible to prepare a composition used according to the invention when the interferential goniochromatic pigment is incompatible with the hydrophilic continuous phase. Thus, the method for producing the composition used according to the invention is particularly useful when the interferential goniochromatic pigment comprises at least one layer comprising aluminium, for example an aluminium-based layer, that is to say a layer mainly consisting of Al.

The composition used according to the invention can moreover comprise an additional particulate phase which can be present in a proportion of 0 to 30%, preferably of 0.01 to 25%, more preferably still of 0.05 to 20%, by weight with respect to the total weight of the composition, and which can comprise at least one pigment other than the interferential goniochromatic pigment according to the invention and/or at least one pearlescent agent and/or at least one filler used conventionally in cosmetic compositions.

Advantageously, such an addition of particulate phase can make possible better control of the colour path across the composition comprising the goniochromatic pigment with an interferential multilayer structure, so as to avoid some shades which can appear as unsightly to some users (such as green, for example, for users of foundation under conventional conditions) or so as to enhance the range of shades provided to the users starting from the same goniochromatic pigment with an interferential multilayer structure, and thus to better control the volume effect. Advantageously, such an addition of particulate phase makes it possible to reduce the manufacturing cost of the cosmetic composition used according to the invention as, currently, the cost of goniochromatic pigments is high.

The term “pigments other than the interferential goniochromatic pigment of the composition used according to the invention” should be understood as meaning white or coloured and inorganic or organic particles which are insoluble in the liquid hydrophilic phase and which are intended to colour and/or opacify the composition. Such pigments are generally monocolours. The term “fillers” should be understood as meaning colourless or white, inorganic or synthetic and lamellar or nonlamellar particles. The term “pearlescent agents” should be understood as meaning iridescent particles, in particular produced by certain shellfish in their shells or else synthesized. These fillers and pearlescent agents are used in particular to modify the texture of the composition.

The pigments other than the interferential goniochromatic pigment of the composition used according to the invention can be present in the composition in a proportion of 0 to 5%, preferably of 0.01 to 5%, by weight with respect to the total weight of the composition. Above 5% by weight, their presence can mask the effect of the interferential goniochromatic pigment according to the invention, which is unacceptable. The said pigment is chosen from the group consisting of the examples mentioned below. Mention may be made, as inorganic pigments which can be used according to the invention, of titanium, zirconium or cerium oxides, as well as zinc, iron or chromium oxides, ferric blue, and their mixtures. Mention may be made, among organic pigments which can be used according to the invention, of carbon black and barium, strontium, zirconium, calcium or aluminium lakes, the diketopyrrolopyrroles (DPPs) disclosed in the documents EP-A-542 669, EP-A-787 730, EP-A-787 731 and WO 96/08537, and their mixtures.

The pearlescent agents can be present in the composition in a proportion of 0 to 20%, preferably of 0.01 to 15%, by weight with respect to the total weight of the composition. For example, the pearlescent agent which can be used in the invention is chosen from the group consisting of mica covered with titanium oxide, with iron oxide, with natural pigment or with bismuth oxychloride, such as coloured titanium-coated mica, and their mixtures.

The fillers used can be present in a proportion of 0 to 30%, preferably of 0.5 to 15%, by weight with respect to the total weight of the composition. For example, the filler which can be used according to the present invention can be chosen from the group consisting of talc, zinc stearate, mica, kaolin, powders formed from nylon (in particular Orgasol powders) and from polyethylene, Teflon, starch, boron nitride, microspheres formed from copolymers, such as Expancel (Nobel Industrie), Polytrap (Dow Corning), microspheres formed from poly(methyl methacrylate), silicone resin microbeads (Tospearl from Toshiba, for example), and their mixtures.

The composition used according to the invention can furthermore comprise at least one of the ingredients conventionally used in the fields under consideration and more especially in the cosmetic and dermatological fields. These ingredients are chosen in particular from preservatives, thickeners for the aqueous phase (polysaccharide biopolymers, synthetic polymers) or for the fatty phase, fragrances, hydrophilic active principles (moisturizers, for example chosen from the group consisting of water and polyhydric alcohols having from 2 to 8 carbon atoms and from 2 to 6 hydroxyl functional groups, for example chosen from ethylene glycol, glycerol, 1,2-propanediol, diglycerol, erythritol, arabitol, adonitol, sorbitol, dulcitol and D-panthenol) or lipophilic active principles (for example chosen from the group consisting of lanolin and UV-A or B screening agents), antioxidants, colorants, essential oils, plant extracts, vitamins and their derivatives (such as vitamins A, B, C and E), sphingolipids (ceramides), fat-soluble polymers, in particular hydrocarbon-based polymers (such as polybutene, polyalkylenes, polyacrylates and silicone polymers compatible with fatty substances), and their mixtures. The amounts of these various ingredients are those conventionally used in the fields under consideration, for example from 0 to 20%, preferably from 0.01 to 15%, by weight with respect to the total weight of the composition. The nature of these ingredients and their proportion must be compatible with the production of compositions with goniochromatic properties according to the invention.

This composition can have the appearance of a cream, pomade, fluid lotion, soft paste with a dynamic viscosity at 25° C. of the order of 1 to 40 Pa·s, ointment, or cast or moulded solid, in particular in a stick or in a dish.

The composition used according to the invention can advantageously be used for making up the skin (including the lips) and/or superficial body growths, that is to say, in this instance, the skin, including the lips, of the face and of the human body, the hair, the eyelashes or the eyebrows, depending upon the nature of the constituents used. In particular, this composition can be a lip lacquer or a lip gloss which can be used as such or for applying to a lipstick film, in particular for enhancing its volume effect (pulping). It can also constitute a liquid or solid foundation, a concealer or product for the outlines of the eyes, an eyeliner, a mascara or an eyeshadow. More specifically, a subject-matter of the invention is a lip product, a foundation or a mascara.

Another subject-matter of the invention is a method for the cosmetic treatment of the skin, the hair, the eyelashes and the eyebrows, which comprises the application, to the skin (including the lips) and/or the superficial body growths, that is to say, in this instance, the skin, including the lips, of the face and of the human body, the hair, the eyelashes or the eyebrows, which comprises the use according to the invention.

A further subject-matter of the invention is a method for making up the skin (including the lips) and/or the superficial body growths, that is to say, in this instance, the skin, including the lips, of the face and of the human body, the hair, the eyelashes or the eyebrows, which comprises the application, to the skin, the hair, the eyelashes and the eyebrows, which comprises the use according to the invention.

Apart from the specific method of production described above, which can be more particularly used for compositions containing at least one goniochromatic pigment with an interferential multilayer structure comprising at least one aluminium-based layer, the composition used according to the invention can be obtained according to the methods of preparation conventionally used in the cosmetic or dermatological fields.

The composition examples below are given by way of illustration of the invention and without a limiting nature.

### EXAMPLE 1

### O/W Type Foundation

An oil-in-water type emulsion was prepared in a manner well known to persons skilled in the art, based on the following composition:

| | |
|---|---|
| Apricot stone oil | 9 g |
| Cyclohexadimethylsiloxane | 9 g |
| Thickening agent | 3 g |
| Stearic acid | 2 g |
| Triethanolamine | 1 g |
| Glyceryl mnodistearate/stearic acid/glycerol | 2.9 g |
| Glycerol | 5 g |
| Xanthan gum | 0.3 g |
| Sodium pyrrolidonecarboxylate | 1.5 g |
| Interferential goniochromatic pigment: | 10 g |
| Sicopearl Fantastico Or from BASF | |
| Preservatives | q.s. |
| Water | q.s. for 100 g |

A foundation is obtained which has a yellow/green colour and which exhibits a volume effect once applied to the skin, for example of the face.

In the same way, by replacing the interferential goniochromatic pigment Sicopearl Fantastico Or with an interferential goniochromatic pigment Sicopearl Fantastico Rose from BASF, prepared, for its part, according to the method of manufacture according to the invention, a foundation is obtained which has a pink/brown-pink colour and which exhibits a volume effect once applied to the skin, for example of the face.

In the same way, by replacing the interferential goniochromatic pigment Sicopearl Fantastico Or or Rose with an interferential goniochromatic pigment Sicopearl Fantastico Ruby from BASF, prepared, for its part, according to the method of manufacture according to the invention, a foundation is obtained which has a red/orange colour and which exhibits a volume effect once applied to the skin, for example of the face.

### EXAMPLE 2

### Mascara

A mascara having the following composition (in % by weight) was prepared:

| | |
|---|---|
| Stearic acid | 6.00 |
| Glyceryl stearate | 3.70 |
| Beeswax | 5.50 |
| Carnauba wax | 1.90 |
| Paraffin | 7.50 |
| Rosin | 1.80 |
| Ethylparaben | 0.04 |
| Propylparaben | 0.03 |
| Interferential goniochromatic pigment: | 5.00 |
| Sicopearl Fantastic Ruby from BASF | |
| Methylparaben | 0.23 |
| Triethanolamine | 3.00 |
| Hydroxyethyl cellulose | 0.20 |
| Ethoxydiglycol | 0.02 |
| Water | q.s. for 100 |

When the composition according to the invention is applied directly to black eyelashes, a very intense green make up is obtained which becomes blue according to the angle of observation.

### EXAMPLE 3

### Eyeliner

An eyeliner was prepared having the following composition:

| | |
|---|---|
| Polyvinyl alcohol | 0.4 g |
| (Rhodoviol 4/125 from RHODIA CHIMIE) 0 | |
| Hydroxypropyl cellulose | 0.015 g |
| (Klucel H from the company HERCULES) | |
| Vinylpyrrolidone/vinyl acetate copolymer | 0.9 g |
| (70/30) in ethanol at 50% (Luviskol VA73E | |
| from BASF) | |
| Poly(para-hydroxystyrene) | 2.5 g |
| (“PHS-N from the company CLARIANT) | |
| Ethyl acrylate/methyl methacrylate copolymer | 2.5 g |
| (8/2) in aqueous dispersion | |
| (Daitosol 5000 AD from DAITO) | |
| Propylene glycol | 14.4 g |
| Ethanol | 5.2 g |
| Oxyethylenated oleyl alcohol (20 EO) | 4 g |
| Magnesium aluminium silicate hydrate | 2 g |
| Interferential goniochromatic pigment: | 5 g |
| Sicopearl Fantastico Ruby from BASF | |
| Black pigments | 15 g |
| Preservatives | q.s. |
| Water | q.s for 100 g |

An eyeliner is obtained which is black in colour, with yellow to green highlights, depending on the angle of the light, and which exhibits a volume effect.

### EXAMPLE 4

### Lip Lacquer

A fluid lipstick is prepared according to European Patent Application EP-A1-0 935 960 having the following composition:

| | |
|---|---|
| Interferential goniochromatic pigment: | 4 g |
| Sicopearl Fantastic Or from BASF | |
| 1,2-Pentanediol | 3 g |
| Ethyl alcohol | 5 g |
| Sodium methyl p-hydroxybenzoate | 0.4 g |
| Aqueous dispersion of polyurethane containing | q.s. for 100 g |
| 49% of solid matter, sold under the | |
| name “SANUCURE 2255” by the company | |
| SANCOR | |

A lip lacquer is obtained which is yellow to green in colour, depending upon the angle of the light, and which exhibits a volume effect once applied to the lips.

In the same way, by replacing the interferential goniochromatic pigment Sicopearl Fantastico Rose with an interferential goniochromatic pigment Sicopearl Fantastico Ruby from BASF, a lip lacquer is obtained which is pink to brown-pink in colour, depending upon the angle of the light, and which exhibits a volume effect once applied to the lips.

1. Use of an interferential goniochromatic pigment in a cosmetic composition in order to give a volume effect, the said cosmetic composition containing a hydrophilic continuous phase and comprising at least one goniochromatic pigment with an interferential multilayer structure and a cosmetically acceptable medium containing a hydrophilic continuous phase, the said medium comprising at least one hydrophilic substance, which is at least partially miscible with or soluble in water, which may be liquid, pasty or solid at ambient temperature and at atmospheric pressure.

2. Use according to claim 1, the said composition being such that it comprises from 0.01 to 5%, of pigment other than the goniochromatic pigment with an interferential multilayer structure, by weight with respect to the total weight of the composition.

3. Use according to either of claims 1 and 2, such that the multilayer structure comprises at least two layers, each layer being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

4. Use as a make-up according to one of claims 1 to 3.

5. Use according to one of claims 1 to 4, such that the interferential multilayer structure is chosen from the group consisting of the structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MoS₂; Fe₂O₃/SiO₂/mica-oxide/SiO₂/Fe₂O₃.

6. Use according to one of claims 1 to 5, such that the said interferential pigment is incorporated in the said composition in an amount of 0.01 to 50%, preferably of 0.5 to 25%, by weight with respect to the total weight of the said composition.

7. Use according to one of claims 1 to 6, which is provided in the form of a product to be applied to the skin, including the lips, both of the human body and of the face, the hair, the eyelashes and the eyebrows.

8. Use according to one of claims 1 to 7, such that the said medium contains water or a mixture of water and at least one hydrophilic organic solvent and represents from 0.5 to 99.99% of the total weight of the said composition.

9. Use according to claim 8, such that the said hydrophilic organic solvent is chosen from the group consisting of linear or branched lower monoalcohols having from 2 to 5 carbon atoms and polyols.

10. Use according to either of claims 8 and 9, such that the said organic solvent is chosen from the group consisting of ethanol, propanol, glycerol, diglycerol, propylene glycol, sorbitol, panthenol, pentylene glycol, polyethylene glycols and their mixtures.

11. Use according to one of claims 1 to 10, such that the said continuous phase additionally comprises at least one hydrophilic C₂ ether and at least one hydrophilic C₂-C₄ aldehyde.

12. Use according to one of claims 1 to 11, such that the said medium comprises or is provided in the form of a suspension, dispersion or solution in water or an aqueous/alcoholic medium; oil-in-water (O/W) or multiple (W/O/W) emulsion; aqueous or aqueous/alcoholic gel or hydrophilic foam; emulsified gel; dispersion of vesicles; two-phase or multiphase lotion; or spray.

13. Use according to claim 12, such that the said medium comprises or is provided in the form of an oil-in-water (O/W) or multiple (W/O/W) emulsion and that the said composition additionally comprises from 0 to 30%, preferably from 0.01 to 30%, by weight of surfactant with respect to the total weight of the said composition.

14. Use according to one of claims 1 to 13, such that the said composition additionally comprises at least one film-forming polymer.

15. Use according to one of claims 1 to 14, such that the said composition also comprises a fatty phase.

16. Use according to claim 15, such that the said fatty phase contains at least one fatty substance which is liquid at ambient temperature and at atmospheric pressure and/or at least one fatty substance which is solid at ambient temperature and at atmospheric pressure.

17. Use according to claim 16, such that the said fatty substance which is liquid at ambient temperature and at atmospheric pressure is chosen from the group consisting of hydrocarbon-based oils of animal origin; vegetable hydrocarbon-based oils; linear or branched hydrocarbons; liquid paraffins and their derivatives; synthetic esters and ethers; polyol esters; fatty alcohols having from 12 to 26 carbon atoms; heptanoates, octanoates or decanoates of fatty alcohols; fluorinated oils which are partially hydrocarbon-comprising and/or silicone-comprising; silicone oils, such as polymethylsiloxanes (PDMSs); and their mixtures.

18. Use according to either of claims 16 and 17, such that the said fatty substance which is liquid at ambient temperature and at atmospheric pressure is chosen from the group consisting of perhydrosqualene, triglycerides of heptanoic acid or octanoic acid, sunflower, maize, soybean, grape seed, sesame, apricot, macadamia, castor or avocado oils, triglycerides of caprylic/capric acids, jojoba oil, karite butter oil, petroleum jelly, polydecenes, hydrogenated polyisobutene, isododecane, Purcellin oil,.isopropyl myristate, 2-ethylhexyl palmitate, 2-octyldodecyl stearate, 2-octyldodecyl erucate, isostearyl isostearate, isononyl isononanoate, isostearyl lactate, octyl hydroxystearate, octyldodecyl hydroxystearate, diisostearyl malate, triisocetyl citrate, propylene glycol dioctanoate, neopentyl glycol diheptanoate, diethylene glycol diisononanoate, pentaerythritol esters, octyldodecanol, 2-butyloctanol, 2-hexyldecanol, 2-undecylpentadecanol, oleyl alcohol, cyclomethicones, dimethicones, phenyl trimethicones, phenyltrimethyl-siloxydiphenylsiloxanes, diphenylmethyldimethyltri-siloxanes, diphenyl dimethicones, phenyl dimethicones, polymethylphenylsiloxanes and their mixtures.

19. Use according to one of claims 16 to 18, such that the said fatty substance which is liquid at ambient temperature and at atmospheric pressure represents from 0 to 90%, preferably from 0.01 to 85%, by weight with respect to the total weight of the fatty phase.

20. Use according to one of claims 16 to 19, such that the said fatty substance which is solid at ambient temperature and atmospheric pressure is chosen from the group consisting of waxes, pasty fatty substances, gums and their mixtures.

21. Use according to claim 20, such that the said fatty phase comprises from 0 to 50%, preferably from 0.01 to 40%, more preferably still from 0.1 to 30%, by weight with respect to the total weight of the composition, of at least one compound chosen from the group consisting of waxes, pasty fatty substances and gums.

22. Use according to claim 21, such that the said compound is chosen from the group consisting of beeswax, carnauba wax, candelilla wax, paraffin wax, microcrystalline waxes, ceresin, ozokerite, synthetic waxes, silicone waxes, PDMSs with a high molecular weight, cellulose gums and polysaccharides.

23. Use according to one of claims 1 to 22, such that the said composition moreover comprises an additional particulate phase in a proportion of 0 to 30%, preferably of 0.01 to 20%, more preferably still of 0.05 to 20%, by weight with respect to the total weight of the said composition.

24. Use according to claim 23, such that the said particulate phase comprises at least one pigment other than the goniochromatic pigment with an interferential multilayer structure and/or at least one pearlescent agent and/or at least one filler.

25. Use according to claim 23, such that the said pigment is chosen from the group consisting of titanium, zirconium and cerium oxides, as well as zinc and chromium oxides, ferric blue, carbon black, barium, strontium, zirconium, calcium and aluminium lakes, diketopyrrolopyrroles (DPPs) and their mixtures.

26. Use according to claim 25, such that the said composition comprises from 0 to 20%, preferably from 0.01 to 15%, of pearlescent agent, by weight with respect to the total weight of the composition.

27. Use according to claim 26, such that the pearlescent agent is chosen from the group consisting of mica covered with titanium oxide, with iron oxide, with natural pigment or with bismuth oxychloride, and their mixtures.

28. Use according to one of claims 1 to 27, such that the said composition comprises from 0 to 30%, preferably from 0.5 to 15%, of filler, by weight with respect to the total weight of the composition.

29. Use according to claim 28, such that the filler is chosen from the group consisting of talc, zinc stearate, mica, kaolin, powders formed from nylon and from polyethylene, Teflon, starch, boron nitride, microspheres formed from copolymers, Polytrap and silicone resin microbeads.

30. Use according to one of claims 1 to 29, such that the said composition additionally comprises from 0 to 20%, preferably from 0.01 to 15%, by weight with respect to the total weight of the composition, of at least one additional ingredient.

31. Use according to claim 30, such that the additional ingredient is chosen from the group consisting of preservatives, thickeners for the aqueous phase or for the fatty phase, fragrances, hydrophilic or lipophilic active principles, antioxidants, colorants, essential oils, plant extracts, vitamins and their derivatives, sphingolipids, fat-soluble polymers and their mixtures.

32. Use according to either of claims 31 and 32, such that the additional ingredient is chosen from the group consisting of ethylene glycol, glycerol, 1,2-propanediol, diglycerol, erythritol, arabitol, adonitol, sorbitol, dulcitol, D-panthenol, lanolin, UV-A or B screening agents, vitamins A, B, C and E and their derivatives, polybutene, polyalkylenes, polyacrylates and silicone polymers compatible with fatty substances.

33. Use according to one of claims 1 to 32, of a composition chosen from the group consisting of lip lacquers, lip glosses, liquid or solid foundations, concealers or products for the outline of the eyes, eyeliners, mascaras and eyeshadows.

34. Method for the cosmetic treatment of the skin, the hair, the eyelashes and the eyebrows, which comprises the application to the skin (including the lips) and/or the superficial body growths, that is to say, in this instance, the skin, including the lips, of the face and of the human body, the hair, the eyelashes or the eyebrows, which comprises the use according to one of claims 1 to 33.

35. Method for making up the skin (including the lips) and/or the superficial body growths, that is to say, in this instance, the skin, including the lips, of the face and of the human body, the hair, the eyelashes or the eyebrows, which comprises the application to the skin, the hair, the eyelashes or the eyebrows, which comprises the use according to one of claims 1 to 33.
